(19) United States
(12) Patent Application Publication
(10) Pub. No.: US 2006/0057186 A1
(43) Pub. Date: Mar. 16, 2006

(54) Oral contraceptive multivitamin compound and methods of administration

(76) Inventor: Margaret Heller, New York, NY (US)

Correspondence Address: JENNIFER MEREDITH;MEREDITH & KEYHANI, PLLC, 330 MADISON AVE., 6TH FLOOR, NEW YORK, NY, 10017, US

(21) Appl. No.: 10/938,977

(22) Filed: Sep. 11, 2004

(51) Int. Cl. A61K 36/16; A61K 47/00; A61K 31/56; A61K 33/32

(52) U.S. Cl. 424/439; 514/171; 424/641; 424/752

(57) ABSTRACT

A pharmaceutical preparation comprising progestin, estrogen and a multivitamin agent. The progestin may be between 0 and 0.50 mg Desogesterel and the estrogen may be between 0 and 0.2 mg Ethinyl Estradiol. The multivitamin agent may be any combination of alpha carotene, beta carotene, biotin, bioflavonoid, calcium, chasteberry fruit, chromium, copper, coenzyme Q10, cryptoxanthin, dong quai root, folic acid, ginkgo biloba, garlic, grape seed extract, green tea extract, hesperedin, iodine, iron, lutein, malic acid, manganese, magnesium, milk thistle, molybdenum, niacin, panthothenic acid, potassium, pyridoxine HCL, quercetin, riboflavin, rutan, selenium, thiamine, vitamin A, vitamin B2, vitamin B6, vitamin B12, vitamin C, vitamin D, vitamin E, vitamin K, zinc, zoexanthin and any combination thereof.

### BACKGROUND OF THE INVENTION

Oral contraceptives are the most effective way to prevent pregnancy (99% effective), but have been known to create certain nutrient deficiencies. An increased intake of Vitamin B6, Vitamin B12, Folic acid, Vitamin E, Vitamin C, and Zinc is recommended to prevent the loss of these essential nutrients. Even without accounting for the depletion issues created by oral contraceptive pills, it is especially important for women to supplement their diets with these vitamins along with others such as Vitamin A, Vitamin B2, Iron, Selenium, and Vitamin D. Certain minerals have also been shown to provide health benefits for women.

The former method for maintaining the body's nutritional level while taking Oral Contraceptive pills was to take vitamin supplements. However, many women using Oral Contraceptives do not take extra vitamins either because they are unaware of the depletion issues or because it is bothersome to remember to take multiple pills every day. Furthermore, vitamins are not absolutely essential to health the way Oral Contraceptive pills are essential for preventing pregnancy. For this reason, the importance of vitamins is often overlooked even though studies have reported an increased need for supplementary vitamins in women using OC pills.

The manner in which vitamins are packaged may be another issue affecting women's decision to take vitamins. Portability plays a central role in remembering to take a pill every day. Unlike Oral Contraceptive pills, most people do not carry their daily vitamins around with them because they are not usually conveniently packaged. Pill bottles are often bulky and may not fit in a woman's pocket or small purse.

The most common Oral Contraceptive pills are to be taken once a day. They are made very small so that they are easy to swallow. The pills are typically packaged in small, convenient blister packages. This design labels the days of the week above the pills so that the consumer knows when to take which pill. The pills can also be carried around with the consumer, which makes it easier to remember to take a pill each day.

Generally, OC pills are made up of estrogen, progestin, or some combination of the two. The pills are taken orally over a period of 28 days and contain varying amounts of ingredients according to when they are taken during the month. Usually, from day 1-21 the pill contains estrogen and progestin and from days 22-28 the pill are inactive. Another specific type of OC pill, Mircette, combines Desogestrel (Progestin) with Ethinyl Estradiol (Estrogen). From day 1-21 the pills contain 0.15 mg of Desogestrel and 0.02 mg of Ethinyl Estradiol, for days 22-23 the pills are filled with inactive ingredients and from days 24-28 the pills contain 0.01 mg of Ethinyl Estradiol and no Desogestrel.

U.S. Pat. No. 6,667,050 issued to Boissonealut et al. entitled “Chewable oral Contraceptive” discloses a chewable palatable oral contraceptive tablet comprising an oral contraceptive agent, a chewable carrier suitable for human consumption, and not comprising a ferrocene compound as well as a method of human female oral contraception. The '050 patent however does not disclose the addition of any multi vitamins, minerals or other supplements to combat the vitamin deficiencies associated with taking oral contraceptives.

U.S. Pat. No. 6,190,693 issued to Kaffrissen et al. entitled “Pharmaceutical methods of delivering folic acid” discloses a method of administering folic-acid containing pharmaceutical compositions comprising either an oral contraceptive agent or other hormone replacement composition. Although the '693 patent does provide a method for administering folic acid it does not disclose an oral contraceptive compound comprising multi vitamins, minerals or other supplements to combat vitamin deficiencies associated with taking oral contraceptives.

The present invention describes a new OC pill that makes up for much of the vitamin depletion associated with current OC use. It combines essential vitamins and minerals with a standard oral contraceptive pill, which many women rely on currently to prevent pregnancy. It also makes the job of taking multiple pills each day much more convenient. A compound tablet with OC agents and multi-vitamins will serve as an optimal way to control pregnancy as well as provide vital nutrients for the body.

The present invention combines the agents of progestin and estrogen with a variety of nutrients. These include Vitamin B2, Vitamin B6, Vitamin B12, Folic acid, Vitamin E, Vitamin C, Hesperetin, Vitamin A, Vitamin D, Selenium, Zinc, Iron, Ginkgo, and Green Tea Extract. The present invention will be a pill that is small in size, yet still carries the optimal combination of ingredients. The small size will maintain the current oral contraceptive pills' desired effect in portability and ease of ingestion. The pills will be held in blister packages that outline for the consumer what day of the week to take each pill. A smooth coating shall be applied to assist in swallowing and eliminate the possibility of it dissolving in the mouth.

### SUMMARY OF THE INVENTION

The present invention relates generally to birth control pills and more specifically to birth control pills that are fortified with vitamins.

According to one embodiment of the present invention, a pharmaceutical preparation is disclosed. The pharmaceutical preparation comprising an oral contraceptive agent and a multivitamin agent.

According to another embodiment of the present invention, a pharmaceutical preparation is disclosed comprising progestin, estrogen and a multivitamin agent.

According to another embodiment of the present invention, a pharmaceutical preparation is disclosed comprising an oral contraceptive agent and multivitamin agent. The oral contraceptive agent comprising progestin and estrogen and the multivitamin agent comprising between 1.0 and 2.0 mg Vitamin B2 (riboflavin), between 1.0 and 3.0 mg Vitamin B6, between 4.0 and 8.0 mg Vitamin B12, Hesperetin, between 200 and 600 mcg Folic Acid, between 10 and 40 IU Vitamin E, between 50 and 70 mg Vitamin C, between 20 and 40 mcg Selenium, between 200 and 600 IU Vitamin D, between 2000 and 4000 IU Vitamin A, between 50 and 150 mg Ginkgo, green tea extract, and between 10 and 20 mg Zinc.

According to yet another embodiment, a pharmaceutical preparation formulated as 28 tablets is disclosed. The 28 tablets consist of a first pharmaceutical preparation for days 1-21, wherein the first pharmaceutical preparation contains progestin, estrogen and a multivitamin agent; and a second pharmaceutical preparation for days 22-28, wherein the second pharmaceutical preparation is comprised of a multivitamin agent.

According to yet another embodiment, a pharmaceutical preparation formulated as 28 tablets is disclosed as follows: a first pharmaceutical preparation for days 1-21, wherein the first pharmaceutical preparation contains progestin, estrogen and a first multivitamin agent; and a second pharmaceutical preparation for days 22-28, wherein the pharmaceutical preparation is comprised of a second multivitamin agent.

These and other features, aspects and advantages of the present invention will become better understood with reference to the description and claims.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

A pharmaceutical preparation, comprising an oral contraceptive agent and a multivitamin agent. The oral contraceptive agent may be any oral contraceptive (OC) known within the art. OC tablets that contain both estrogen and progestin are the most common. The estrogen compound used in most oral contraceptives is estradiol. Many different progestins are used. These may include Norethynodrel, Norethindrone, Norethindrone acetate, Norgestimate, Desogestrel, Ethynodiol diacetate, Norgestrel, Levonorgestrel and Drospirenone. The standard dosages have been OCs preparations with estrogen at dosages of 30/35 mcg, but now low or ultra-low OCs are available with estrogen dosage levels of 20 mcg or less. Brands include the following: Desogestrel/estradiol (Desogen, Mircette, Ortho-Cept) Low-dose contraceptive. Drospirenone/estradiol (Yasmin) Low-dose contraceptive. Drospirenone has effects similar to natural progesterone. Agent may have fewer side effects than older OCs, including weight gain and improved sense of well being. Ethynodiol/estradiol (Demulen, Zovia). Gestodene/estradiol (Minesse). Very promising low-dose oral contraceptive taken in a 24-day pack. (Approved in Europe.) Levonorgestrel/estradiol (Alesse, Levlen, Levlite, Nordette, Tri-Norinyl, Triphasil). Low-dose contraceptive. Norethindrone acetate/estradiol (Estrostep, Loestrin). Norethindrone/estradiol (Brevicon, Estrostep, Genora, Intercon, Jenest, ModiCon, Necon, N.E.E. Nelova, Norethrin, Norinyl, Ortho-Novum, Ovcon, Tri-Norinyl). Norethindrone/ Mestranol (Intercon, Necon, Nelova, Norethrin, Ortho-Novum). Norgestimate/estradiol (Ortho-Cyclen, Ortho Tri-Cyclen). Tri-Cyclen only OC to date approved by the FDA for treatment of acne. Norgestrel/estradiol (Lo/Ovral).

Combination pills are sold in 21-day or 28-day packs: Each pill in the 21-day pack contains the necessary estrogen and progestin. The 28-day pack adds seven differently colored “reminder” pills; they are inactive and do not contain hormones, but help the user maintain her daily routine during seven days between active pill use. However, the present invention provides vitamin supplement during the 7 day inactive period.

OCs may be taken in cycles that include pills of the same or different strengths. These are categorized as monophasic (one-phase), biphasic (two-phase) or triphasic (three-phase). A Monophasic schedule is a 21-day pack that uses tablets that are one strength and one color for 21 days. (A 28-day pack adds seven inactive tablets of a different color.) A Biphasic schedule is a 21-day pack consists of tablets of one strength and color taken for 7 or 10 days, then a second tablet with a different strength and color for the next 11 or 14 days. (And a 28-day pack adds seven inactive tablets of a third color.) A Triphasic schedule pack consists of tablets with three different colors and strengths. In the first phase, there are tablets of one color for five to seven days; for phase two, a second color and strength tablets is taken for five to seven days; and for phase three, a third color and strength tablet is taken for 5 to 10 days. The difference in duration of each phase depends on the brand. (And a 28-day pack includes a fourth color inactive tablet for the last seven days.)

There are also progestin-only oral contraceptives. Progestin-only pill brands including the following: Levonorgestrel (Plan B), Norethindrone (Micronor, Nor-QD) and Norgestrel (Ovrette). Progestin-only pills, which only contain progestins, are always sold in 28-day packs and all the pills are active. Progestin-only pills must be taken at precisely the same time each day to maintain top effectiveness. If a woman deviates from her pill schedule for even three hours, she should call her doctor about using back-up contraception for the next two days. Progestin-only pill users will experience even lighter periods than those taking combination pills; some may not have periods at all. According to the present invention, progestin only pills would have the same birth control ingredients for all 28 days. That is, days 1-21 and days 22-28, would all have an oral contraceptive agent and a multivitamin agent. However, the multivitamin may very according to the day.

In all cases, women continue to menstruate, but their periods are lighter, shorter, more regular, and less painful than bleeding in women who are not on the pill. Typically, the user takes the first pill either on the Sunday after her period starts or during the first 24 hours of her period. The remaining pills are taken once a day, ideally at the same time of day, until the pack is used up. Previously, the user, if she has a 21-day pack, waits seven days before starting a new pack. If she is on the 28-day pack, she takes the seven inactive pills. According to the present invention, the user would take seven pills containing multivitamin agents only. In this manner, the user only has to remember to take their pill everyday, rather than remembering on and off days and taking vitamins.

As discussed, there are numerous types and kinds of oral contraceptives. However, the most commonly prescribed are 28 days and comprised of an estrogen and a progestin. The estrogen may be Ethinyl Estradiol, which according to a preferred embodiment may be between 0 and 0.20 mg. The estrogen may also be Mestranol. The progestin may be Norethynodrel, Norethindrone, Norethindrone acetate, Norgestimate, Desogestrel, Ethynodiol diacetate, Norgestrel, Levonorgestrel and Drospirenone. According to one embodiment, the progestin is between 0 and 0.50 mg Desogesterel.

The multivitamin agent may be selected from the group consisting of alpha carotene, beta carotene, biotin, bioflavonoid, calcium, chasteberry fruit, chromium, copper, coenzyme Q10, cryptoxanthin, dong quai root, folic acid, ginkgo biloba, garlic, grape seed extract, green tea extract, hesperedin, iodine, iron, lutein, malic acid, manganese, magnesium, milk thistle, molybdenum, niacin, panthothenic acid, potassium, pyridoxine HCL, quercetin, riboflavin, rutan, selenium, thiamine, vitamin A, vitamin B2, vitamin B6, vitamin B12, vitamin C, vitamin D, vitamin E, vitamin K, zinc, zoexanthin and any combination thereof. According to a preferred embodiment, the Vitamin B6 is between 0.1 and 2 mg. The Vitamin B12 may be between 1 and 6 mcg. The Folic acid may be between 1 and 400 mcg. The Vitamin E may be between 1 and 300 IU. The Vitamin C may be ascorbyl palmitate and be between 1 and 60 mg. There may be between 1 and 3000 IU of Vitamin A. There may be between 1 and 400 IU of Vitamin D. There may be between 1 and 20 mcg of Selenium. There may be between 1 and 15 mg of Zinc. There may be between 1 and 10 mg of Iron. There may be between 1 and 1.5 mg of Vitamin B12. There may be between 1 and 100 mg of Ginko. There may be between 1,000 and 1,500 milligrams of calcium. There may be between 150 and 250 milligrams of magnesium. The tablet may contain a sucrose powder which acts as a binding agent. There may also be a smooth coating assisting swallowing, a sugar based coating and/or an enteric coating. It should also be understood that the term multivitamin agent may include vitamins, minerals and herbal supplements.

The American Journal of Obstetrics and Gynecology reported a study which found that 1,200 milligrams of calcium taken daily reduced the physical and psychological symptoms associated with PMS by almost 50 percent. Another study reported in the Journal of Women's Health found that 200 milligrams of magnesium a day produced a 40 percent reduction in fluid retention, breast tenderness and bloating.

The preparation may also be in the form of a transdermal administration patch. Oral contraception delivered via injections and skin patches are currently available and reliable. They typically contain both estrogen and progestin and their side effects are similar to the combination OCs. The present invention envisions vitamins and oral contraceptives delivered orally, via injection and transdermally.

According to one embodiment, a pharmaceutical preparation is disclosed comprising an oral contraceptive agent and multivitamin agent. The oral contraceptive agent comprising progestin and estrogen and the multivitamin agent comprising between 1.0 and 2.0 mg Vitamin B2 (riboflavin), between 1.0 and 3.0 mg Vitamin B6, between 4.0 and 8.0 mg Vitamin B12, between 5 and 50 mg Hesperedin, between 200 and 600 mcg Folic Acid, between 10 and 40 IU Vitamin E, between 50 and 70 mg Vitamin C, between 20 and 40 mcg Selenium, between 200 and 600 IU Vitamin D, between 2000 and 4000 IU Vitamin A, between 50 and 150 mg Ginkgo, between 25 and 100 mg green tea extract, and between 10 and 20 mg Zinc.

It is important to remember that within the context of the present invention, there will generally be at least two different pharmaceutical preparations. According to one embodiment, a pharmaceutical preparation is formulated as 28 tablets as follows: a first pharmaceutical preparation for days 1-21, wherein the first pharmaceutical preparation contains progestin, estrogen and a multivitamin agent; and a second pharmaceutical preparation for days 22-28, wherein the pharmaceutical preparation is comprised of a multivitamin agent. In this manner, the user is being delivered their birth control and multivitamins for days 1-21. During the period of days 22-28, in which the pills were previously inactive or the user would not take pills, according to the present invention the user is taking pills that contain only multivitamins. The multivitamin agent is selected from the group consisting of alpha carotene, beta carotene, biotin, bioflavonoid, calcium, chasteberry fruit, chromium, copper, coenzyme Q10, cryptoxanthin, dong quai root, folic acid, ginkgo biloba, garlic, grape seed extract, green tea extract, hesperedin, iodine, iron, lutein, malic acid, manganese, magnesium, milk thistle, molybdenum, niacin, panthothenic acid, potassium, pyridoxine HCL, quercetin, riboflavin, rutan, selenium, thiamine, vitamin A, vitamin B2, vitamin B6, vitamin B12, vitamin C, vitamin D, vitamin E, vitamin K, zinc, zoexanthin and any combination thereof.

The pharmaceutical preparation may be 1.5 mg Vitamin B2 (riboflavin), 2 mg Vitamin B6, 6 mg Vitamin B12, Hesperetin, 400 mcg Folic Acid, 30 IU Vitamin E, 60 mg Vitamin C, 25 mcg Selenium, 10 mg Iron, 400 IU Vitamin D, 3000 IU Vitamin A, 100 mg Ginkgo, green tea extract, and 15 mg Zinc.

It may also be desirable to have different preparations of vitamins for different days. For example, it is well known that during days 22-28 there is typically a loss of iron through menstruation. Because of this it may be desirable to increase the dosage of Iron delivered during these days. As such, days 1-21 would contain between 0 and 10 mg of Iron and days 22-28 would contain between 11 and 30 mg of Iron.

According to one embodiment, a pharmaceutical preparation is formulated as 28 tablets as follows: a first pharmaceutical preparation for days 1-21, wherein the first pharmaceutical preparation contains progestin, estrogen and a first multivitamin agent; and a second pharmaceutical preparation for days 22-28, wherein the pharmaceutical preparation is comprised of a second multivitamin agent.

The first pharmaceutical preparation may be 1.5 mg Vitamin B2 (riboflavin), 2 mg Vitamin B6, 6 mg Vitamin B12, 25 mg Hesperidin, 400 mcg Folic Acid, 30 IU Vitamin E, 60 mg Vitamin C, 25 mcg Selenium, 10 mg Iron, 400 IU Vitamin D, 3000 IU Vitamin A, 100 mg Ginkgo, 25 mg green tea extract, and 15 mg Zinc.

The second pharmaceutical preparation may be 1.5 mg Vitamin B2 (riboflavin), 2 mg Vitamin B6, 6 mg Vitamin B12, 25 mg Hesperidin, 800 mcg Folic Acid, 30 IU Vitamin E, 60 mg Vitamin C, 25 mcg Selenium, 25 mg Iron, 400 IU Vitamin D, 3000 IU Vitamin A, 100 mg Ginkgo, 25 green tea extract, and 15 mg Zinc.

It may also be desirable to deliver vitamins, minerals or herbal treatments during the period in which a woman may experience PMS. In this manner, a different multivitamin agent would be in pills for days 16-21, the typical period during which a woman may experience PMS. This may be disclosed as a pharmaceutical preparation formulated as 28 tablets as follows: a first pharmaceutical preparation for days 1-15, wherein the first pharmaceutical preparation contains progestin, estrogen and a first multivitamin agent; and a second pharmaceutical preparation for days 16-21, wherein the second pharmaceutical preparation contains progestin, estrogen and a second multivitamin agent; and a third pharmaceutical preparation for days 22-28, wherein the pharmaceutical preparation is comprised of a multivitamin agent. The first multivitamin agent, second multivitamin agent and third multivitamin agent may be any combination of vitamins, minerals and supplements selected from the group consisting of alpha carotene, beta carotene, biotin, bioflavonoid, calcium, chasteberry fruit, chromium, copper, coenzyme Q10, cryptoxanthin, dong quai root, folic acid, ginkgo biloba, garlic, grape seed extract, green tea extract, hesperedin, iodine, iron, lutein, malic acid, manganese, magnesium, milk thistle, molybdenum, niacin, panthothenic acid, potassium, pyridoxine HCL, quercetin, riboflavin, rutan, selenium, thiamine, vitamin A, vitamin B2, vitamin B6, vitamin B12, vitamin C, vitamin D, vitamin E, vitamin K, zinc, zoexanthin and any combination thereof.

In this manner, a woman can easily prevent PMS by taking her regular birth control pill which contains herbs that prevent PMS during those days in which she is at risk. Fortunately, several different natural herbs and enzymes have been found to be very beneficial for a woman's endocrine system and can help ease symptoms associated with PMS and menopause. Supplementing the health of the endocrine system with herbs like chasteberry fruit, milk thistle and dong quai root promotes a balanced endocrine system, which is intimately involved with a women's quality of life. A good supply of enzymes such as protease, amylase and lipase also ensure that your body can properly assimilate the active nutrients in these herbs. As such, the second multivitamin agent which is taken on days 16-21 may contain chasteberry fruit, milk thistle and dong quai root.

It should be understood that the foregoing relates to preferred embodiments of the invention and that modifications may be made without departing from the spirit and scope of the invention as set forth in the following claims.

1. A pharmaceutical preparation, comprising an oral contraceptive agent and a multivitamin agent.

2. The pharmaceutical preparation as in claim 1, wherein said oral contraceptive agent is comprised of a progestin and an estrogen.

3. The pharmaceutical preparation as in claim 2, wherein said progestin is selected from the group consisting of Norethynodrel, Norethindrone, Norethindrone acetate, Norgestimate, Desogestrel, Ethynodiol diacetate, Norgestrel, Levonorgestrel and Drospirenone.

4. The pharmaceutical preparation as in claim 2, wherein said estrogen is Ethinyl Estradiol or mestranol.

5. The pharmaceutical preparation as in claim 3, wherein said progestin is between 0 and 0.50 mg Desogesterel.

6. The pharmaceutical preparation as in claim 4, wherein said estrogen is between 0 and 0.2 mg Ethinyl Estradiol.

7. The pharmaceutical preparation as in claim 1, wherein said multivitamin agent is selected from the group consisting of alpha carotene, beta carotene, biotin, bioflavonoid, calcium, chasteberry fruit, chromium, copper, coenzyme Q10, cryptoxanthin, dong quai root, folic acid, ginkgo biloba, garlic, grape seed extract, green tea extract, hesperedin, iodine, iron, lutein, malic acid, manganese, magnesium, milk thistle, molybdenum, niacin, panthothenic acid, potassium, pyridoxine HCL, quercetin, riboflavin, rutan, selenium, thiamine, vitamin A, vitamin B2, vitamin B6, vitamin B12, vitamin C, vitamin D, vitamin E, vitamin K, zinc, zoexanthin and any combination thereof.

8. The pharmaceutical preparation as in claim 7, wherein said Vitamin B6 is between 0.1 and 2 mg.

9. The pharmaceutical preparation as in claim 7, wherein said Vitamin B12 is between 1 and 6 mcg.

10. The pharmaceutical preparation as in claim 7, wherein said Folic acid is between 1 and 400 mcg.

11. The pharmaceutical preparation as in claim 7, wherein said Vitamin E is between 1 and 300 IU.

12. The pharmaceutical preparation as in claim 7, wherein said Vitamin C is ascorbyl palmitate.

13. The pharmaceutical preparation as in claim 7, wherein said Vitamin C is between 1 and 60 mg.

14. The pharmaceutical preparation as in claim 7, wherein said Vitamin A is between 1 and 3000 IU.

15. The pharmaceutical preparation as in claim 7, wherein said Vitamin D is between 1 and 400 IU.

16. The pharmaceutical preparation as in claim 7, wherein said Selenium is between 1 and 20 mcg.

17. The pharmaceutical preparation as in claim 7, wherein said Zinc is between 1 and 15 mg.

18. The pharmaceutical preparation as in claim 7, wherein said Iron is between 1 and 10 mg.

19. The pharmaceutical preparation as in claim 7, wherein said Vitamin B2 is between 1 and 1.5 mg.

20. The pharmaceutical preparation as in claim 7, wherein said Ginko is between 1 and 100 mg.

21. The pharmaceutical preparation as in claim 7, further comprising sucrose powder as a binding agent.

22. The pharmaceutical preparation as in claim 1, further comprising a coating.

23. The pharmaceutical preparation as in claim 7, wherein said calcium is between 1,000 and 1,500 milligrams of calcium. There may be between 150 and 250 milligrams of magnesium.

24. The pharmaceutical preparation as in claim 7, wherein said magnesium is between 150 and 250 milligrams.

25. The pharmaceutical preparation as in claim 1, wherein said preparation is a transdermal administration patch.

26. A pharmaceutical preparation comprising progestin, estrogen and a multivitamin agent.

27. The pharmaceutical preparation as in claim 26, wherein said progestin is selected from the group consisting of Norethynodrel, Norethindrone, Norethindrone acetate, Norgestimate, Desogestrel, Ethynodiol diacetate, Norgestrel, Levonorgestrei and Drospirenone.

28. The pharmaceutical preparation as in claim 26, wherein said is estrogen is between 0 and 0.2 mg Ethinyl Estradiol.

29. The pharmaceutical preparation as in claim 26, wherein said multivitamin agent is selected from the group consisting of alpha carotene, beta carotene, biotin, bioflavonoid, calcium, chasteberry fruit, chromium, copper, coenzyme Q10, cryptoxanthin, dong quai root, folic acid, ginkgo biloba, garlic, grape seed extract, green tea extract, hesperedin, iodine, iron, lutein, malic acid, manganese, magnesium, milk thistle, molybdenum, niacin, panthothenic acid, potassium, pyridoxine HCL, quercetin, riboflavin, rutan, selenium, thiamine, vitamin A, vitamin B2, vitamin B6, vitamin B12, vitamin C, vitamin D, vitamin E, vitamin K, zinc, zoexanthin and any combination thereof.

30. The pharmaceutical preparation as in claim 29, wherein said Vitamin B6 is between 0.1 and 2 mg.

31. The pharmaceutical preparation as in claim 29, wherein said Vitamin B12 is between 1 and 6 mcg.

32. The pharmaceutical preparation as in claim 29, wherein said Folic acid is between 1 and 400 mcg.

33. The pharmaceutical preparation as in claim 29, wherein said Vitamin E is between 1 and 300 IU.

34. The pharmaceutical preparation as in claim 29, wherein said Vitamin C is ascorbyl palmitate.

35. The pharmaceutical preparation as in claim 29, wherein said Vitamin C is between 1 and 60 mg.

36. The pharmaceutical preparation as in claim 29, wherein said Vitamin A is between 1 and 3000 IU.

37. The pharmaceutical preparation as in claim 29, wherein said Vitamin D is between 1 and 400 IU.

38. The pharmaceutical preparation as in claim 29, wherein said Selenium is between 1 and 20 mcg.

39. The pharmaceutical preparation as in claim 29, wherein said Zinc is between 1 and 15 mg.

40. The pharmaceutical preparation as in claim 29, wherein said Iron is between 1 and 10 mg.

41. The pharmaceutical preparation as in claim 26, wherein said Vitamin B2 is between 1 and 1.5 mg.

42. The pharmaceutical preparation as in claim 26, wherein said Ginko is between 1 and 100 mg.

43. The pharmaceutical preparation as in claim 26, further comprising sucrose powder as a binding agent.

44. The pharmaceutical preparation as in claim 26, further comprising a smooth coating assisting swallowing.

45. The pharmaceutical preparation as in claim 26, further comprising a sugar based coating.

46. The pharmaceutical preparation as in claim 26, further comprising an enteric coating.

47. The pharmaceutical preparation as in claim 26, wherein said preparation is a transdermal administration patch.

48. A pharmaceutical preparation comprising an oral contraceptive agent and multivitamin agent, said oral contraceptive agent comprising progestin and estrogen and said multivitamin agent comprising between 1.0 and 2.0 mg Vitamin B2 (riboflavin), between 1.0 and 3.0 mg Vitamin B6, between 4.0 and 8.0 mg Vitamin B12, Hesperetin, between 200 and 600 mcg Folic Acid, between 10 and 40 IU Vitamin E, between 50 and 70 mg Vitamin C, between 20 and 40 mcg Selenium, between 200 and 600 IU Vitamin D, between 2000 and 4000 IU Vitamin A, between 50 and 150 mg Ginkgo, green tea extract, and between 10 and 20 mg Zinc.

49. The pharmaceutical preparation as in claim 48, wherein said progestin is selected from the group consisting of Norethynodrel, Norethindrone, Norethindrone acetate, Norgestimate, Desogestrel, Ethynodiol diacetate, Norgestrel, Levonorgestrel and Drospirenone.

50. The pharmaceutical preparation as in claim 48, wherein said estrogen is between 0 and 0.2 mg Ethinyl Estradiol.

51. The pharmaceutical preparation as in claim 48, further comprising sucrose powder as a binding agent.

52. The pharmaceutical preparation as in claim 48, further comprising a smooth coating assisting swallowing.

53. The pharmaceutical preparation as in claim 48, further comprising a sugar based coating.

54. The pharmaceutical preparation as in claim 48, further comprising an enteric coating.

55. The pharmaceutical preparation as in claim 48, wherein said preparation is a transdermal administration patch.

56. A pharmaceutical preparation formulated as 28 tablets as follows:
a first pharmaceutical preparation for days 1-21, wherein said first pharmaceutical preparation contains progestin, estrogen and a multivitamin agent; and
a second pharmaceutical preparation for days 22-28, wherein said pharmaceutical preparation is comprised of a multivitamin agent.

57. The pharmaceutical preparation as in claim 56, wherein said multivitamin agent is selected from the group consisting of alpha carotene, beta carotene, biotin, bioflavonoid, calcium, chasteberry fruit, chromium, copper, coenzyme Q10, cryptoxanthin, dong quai root, folic acid, ginkgo biloba, garlic, grape seed extract, green tea extract, hesperedin, iodine, iron, lutein, malic acid, manganese, magnesium, milk thistle, molybdenum, niacin, panthothenic acid, potassium, pyridoxine HCL, quercetin, riboflavin, rutan, selenium, thiamine, vitamin A, vitamin B2, vitamin B6, vitamin B12, vitamin C, vitamin D, vitamin E, vitamin K, zinc, zoexanthin and any combination thereof.

58. The pharmaceutical preparation as in claim 56, wherein said pharmaceutical preparation is comprised of 1.5 mg Vitamin B2 (riboflavin), 2 mg Vitamin B6, 6 mg Vitamin B12, Hesperetin, 400 mcg Folic Acid, 30 IU Vitamin E, 60 mg Vitamin C, 25 mcg Selenium, 10 mg Iron, 400 IU Vitamin D, 3000 IU Vitamin A, 100 mg Ginkgo, green tea extract, and 15 mg Zinc.

59. The pharmaceutical preparation as in claim 56, further comprising sucrose powder as a binding agent.

60. The pharmaceutical preparation as in claim 56, further comprising a smooth coating assisting swallowing.

61. The pharmaceutical preparation as in claim 56, further comprising a sugar based coating.

62. The pharmaceutical preparation as in claim 56, further comprising an enteric coating.

63. The pharmaceutical preparation as in claim 56, wherein said preparation is a transdermal administration patch.

64. The pharmaceutical preparation as in claim 56, wherein said progestin is selected from the group consisting of Norethynodrel, Norethindrone, Norethindrone acetate, Norgestimate, Desogestrel, Ethynodiol diacetate, Norgestrel, Levonorgestrel and Drospirenone.

65. The pharmaceutical preparation as in claim 56, wherein said estrogen is between 0 and 0.2 mg Ethinyl Estradiol.

66. A pharmaceutical preparation formulated as 28 tablets as follows:
a first pharmaceutical preparation for days 1-21, wherein said first pharmaceutical preparation contains progestin, estrogen and a first multivitamin agent; and
a second pharmaceutical preparation for days 22-28, wherein said pharmaceutical preparation is comprised of a second multivitamin agent.

67. The pharmaceutical preparation as in claim 66, wherein said first multivitamin agent is selected from the group consisting of alpha carotene, beta carotene, biotin, bioflavonoid, calcium, chasteberry fruit, chromium, copper, coenzyme Q10, cryptoxanthin, dong quai root, folic acid, ginkgo biloba, garlic, grape seed extract, green tea extract, hesperedin, iodine, iron, lutein, malic acid, manganese, magnesium, milk thistle, molybdenum, niacin, panthothenic acid, potassium, pyridoxine HCL, quercetin, riboflavin, rutan, selenium, thiamine, vitamin A, vitamin B2, vitamin B6, vitamin B12, vitamin C, vitamin D, vitamin E, vitamin K, zinc, zoexanthin and any combination thereof.

68. The pharmaceutical preparation as in claim 66, wherein said second multivitamin agent is selected from the group consisting of alpha carotene, beta carotene, biotin, bioflavonoid, calcium, chasteberry fruit, chromium, copper, coenzyme Q10, cryptoxanthin, dong quai root, folic acid, ginkgo biloba, garlic, grape seed extract, green tea extract, hesperedin, iodine, iron, lutein, malic acid, manganese, magnesium, milk thistle, molybdenum, niacin, panthothenic acid, potassium, pyridoxine HCL, quercetin, riboflavin, rutan, selenium, thiamine, vitamin A, vitamin B2, vitamin B6, vitamin B12, vitamin C, vitamin D, vitamin E, vitamin K, zinc, zoexanthin and any combination thereof.

69. The pharmaceutical preparation as in claim 66, wherein first said pharmaceutical preparation is comprised of 1.5 mg Vitamin B2 (riboflavin), 2 mg Vitamin B6, 6 mg Vitamin B12, Hesperetin, 400 mcg Folic Acid, 30 IU Vitamin E, 60 mg Vitamin C, 25 mcg Selenium, 8 mg Iron, 400 IU Vitamin D, 3000 IU Vitamin A, 100 mg Ginkgo, green tea extract, and 15 mg Zinc.

70. A pharmaceutical preparation formulated as 28 tablets as follows:
a first pharmaceutical preparation for days 1-15, wherein said first pharmaceutical preparation contains progestin, estrogen and a first multivitamin agent; and
a second pharmaceutical preparation for days 16-21, wherein said second pharmaceutical preparation contains progestin, estrogen and a second multivitamin agent
a third pharmaceutical preparation for days 22-28, wherein said pharmaceutical preparation is comprised of a multivitamin agent.

71. The pharmaceutical preparation as in claim 70, wherein said multivitamin agent is selected from the group consisting of alpha carotene, beta carotene, biotin, bioflavonoid, calcium, chasteberry fruit, chromium, copper, coenzyme Q10, cryptoxanthin, dong quai root, folic acid, ginkgo biloba, garlic, grape seed extract, green tea extract, hesperedin, iodine, iron, lutein, malic acid, manganese, magnesium, milk thistle, molybdenum, niacin, panthothenic acid, potassium, pyridoxine HCL, quercetin, riboflavin, rutan, selenium, thiamine, vitamin A, vitamin B2, vitamin B6, vitamin B12, vitamin C, vitamin D, vitamin E, vitamin K, zinc, zoexanthin and any combination thereof.
